SEQUENCE LISTING (1 sequence)

SEQ ID NO: 1 - protein, 5 aa, Human
YTPKT

(19) United States
(12) Patent Application Publication
(10) Pub. No.: US 2009/0099095 A1
(43) Pub. Date: Apr. 16, 2009

(54) Novel neuroprotective peptide

(76) Inventors: Peter Eriksson, Vastra Frolunda (SE); Michael Nilson, Vastra Frolunda (SE)

Correspondence Address: LYNN E BARBER, P O BOX 16528, FORT WORTH, TX, 76162, US

(21) Appl. No.: 12/151,814

(22) Filed: May 9, 2008

Related U.S. Application Data
(60) Provisional application No. 60/930,453, filed on May 16, 2007.

(51) Int. Cl. A61K 38/08; C07K 7/00

(52) U.S. Cl. 514/17; 530/330

(57) ABSTRACT

The field of the present invention is a novel neuroprotective peptide, pentinin, having neuroprotective properties. More particularly, the field of the present invention relates to the ability of pentinin (SEQ ID NO: 1) to affect endogenous undifferentiated stem cells to positively modulate neural damage and the use of such peptide for the treatment of disorders of the neural system. The present invention also relates to the manufacture of medicaments, methods of formulation and uses thereof. An intranasal delivery system for administration of pentinin is also described.

### CROSS-REFERENCE TO RELATED APPLICATIONS

This application claims priority from U.S. Provisional Patent Application Ser. No. 60/930,453 filed May 16, 2007.

### FIELD OF THE INVENTION

The field of the present invention is a novel neuroprotective peptide, pentinin, having neuroprotective properties. More particularly, the field of the present invention relates to the ability of pentinin (SEQ ID NO: 1) to affect endogenous undifferentiated stem cells to positively modulate neural damage and the use of such peptide for the treatment of disorders of the neural system. The present invention also relates to the manufacture of medicaments, methods of formulation and uses thereof. An intranasal delivery system for administration of pentinin is also described.

### BACKGROUND OF THE INVENTION

Adult neurogenesis occurs in the dentate gyrus of the hippocampus and in the olfactory bulb. The new neurons arise from adult neural stem/progenitor cells (NSPCs) which reside in the subgranular zone (SGZ) and the subventricular zone (SVZ), respectively¹. These neurons can integrate into pre-existing circuitry, and form active synapses, suggesting a role in basal neuronal replacement². It has also been reported that endogenous or grafted NSPCs are associated with reductions in damage or impairment following various pathological events, including stroke^{3,4}. Although neuronal replacement may be a factor in these instances, data suggest that this may be a minor contribution.

It has been previously recognised that NSPCs may influence the outcomes of pathological events by other means, such as the secretion of various growth factors, including glial-derived neurotrophic factor (GDNF) and nerve growth factor (NGF)⁵. This has been suggested as an advantageous effect of grafting of exogenous NSPCs to areas of damage, as well as for recruited endogenous NSPCs potentially modulating the environment around a lesion. However, in areas where NSPCs reside in close proximity to neurons, such as the dentate gyrus, these endogenous factors could contribute to local neuroprotection.

We chose to study this hypothesis using organotypic hippocampal slice cultures (OHSCs). In such cultures, the architecture of the hippocampal formation remains largely intact, whilst allowing various in vitro manipulations and visualisation of effects on groups of cells within the structure. OHSCs have been used to model various brain pathologies, including stroke and epilepsy. Specifically, the application of glutamate receptor agonists, such as N-methyl-D-aspartic acid (NMDA) and kainite, has been shown to cause excitotoxic injury (the pathological process by which nerve cells are damaged and killed by glutamate and similar substances) in OHSCs, which recapitulates some of the pathophysiology of these disorders. The model, therefore, provides an interesting platform for analysing the interactions between NSPCs and the processes of neurotoxicity and neuroprotection.

This invention discloses the influence of factors produced by adult NSPCs on NMDA-induced excitotoxicity in the hippocampus. We found that medium conditioned by NSPCs provided a significant degree of neuroprotection, and indeed completely abolished NMDA-dependent cell death in the dentate gyrus. In the Cornu Ammonis 1 (CA1) and Cornu Ammonis 3 (CA3) regions of the hippocampus, abolition of neurotoxicity could be achieved by supplementing the conditioned medium (CM), with a very low dose of GDNF.

In order to determine the source of this neuroprotection we reanalysed data from a previous mass spectrometry (MS) study performed in our laboratory. Although we hypothesised that the proteins identified in that study may have been neuroprotective, further analyses, both experimental and in silico, were not promising. A previous study of the inventors of the application herein was designed to find relatively large proteins and we therefore performed a new mass spectrometric analysis of the CM, this time looking for peptides and smaller proteins. These analyses demonstrated that the NSPCs cleave insulin, resulting in a truncated form of the protein and a pentapeptide which we termed pentinin.

We hypothesised that pentinin may have neuroprotective properties, through analogy with glycine-proline-glutamate (GPE), an N-terminal peptide of insulin-like growth factor, which is neuroprotective in different paradigms¹⁴⁻¹⁶. In addition, a C-terminal peptide of mechano-growth factor, a splice variant of IGF-1, has also been shown to be neuroprotective in a NMDA/OHSC model, as well as in vivo¹⁷.

We hypothesised that pentinin was produced in vitro by the cleavage of insulin. This was supported by immunofluorescence of insulin degrading enzyme (IDE) in NSPCs, an enzyme which is known to produce this pentapeptide as a breakdown product¹². IDE has been identified in several subcellular locations, but is primarily cytosolic. Insulin processing usually occurs in endosomes, as part of insulin receptor recycling. Although a proportion is fully degraded by lysosomes, both intact insulin and fragments are secreted by diacytosis¹⁸. Interestingly, it has also been reported that insulin B chain lacking these five residues is fully active, in vitro, suggesting that both fragments may have a role, although it should be noted that IDE further cleaves the B chain to make smaller fragments.

The expression if IDE is not unique to NSPCs. IDE is expressed through the body, in a time and tissue specific manner²⁰.

The blood brain barrier (BBB) is one of the strictest barriers of in vivo therapeutic drug delivery. The barrier is defined by restricted exchange of hydrophilic compounds, small proteins and charged molecules between the plasma and central nervous system (CNS). For decades, the BBB has prevented the use of many therapeutic agents for treating Alzheimer's disease, stroke, brain tumor, head injury, spinal cord injury, depression, anxiety and other CNS disorders. Different attempts were made to deliver the drug across the BBB such as modification of therapeutic agents, altering the barrier integrity, carrier-mediated transport, invasive techniques, etc. However, opening the barrier by such means allows entry of toxins and undesirable molecules to the CNS, resulting in potentially significant damage. An attempt to overcome the barrier in vivo has focused on bypassing the BBB by using a novel, practical, simple and non-invasive approach i.e. intranasal delivery. This method works because of the unique connection which the olfactory and trigeminal nerves (involved in sensing odors and chemicals) provide between the brain and external environments. The olfactory epithelium acting as a gateway for substances entering the CNS and peripheral circulation is well known. The neural connections between the nasal mucosa and the brain provide a unique pathway for the non-invasive delivery of therapeutic agents to the CNS. This pathway also allows drugs which do not cross the BBB to enter the CNS and it eliminates the need for systemic delivery and thereby reducing unwanted systemic side effects. Intranasal delivery does not require any modification of therapeutic agents and does not require drugs to be coupled with any carrier. A wide variety of therapeutic agents, including both small molecules and macromolecules can be rapidly delivered to the CNS using this method²¹

A number of protein therapeutic agents have been successfully delivered to the CNS using intranasal delivery in a variety of species. Neurotrophic factors such as NGF, IGF-I, FGF and ADNF12 have been intranasally delivered to the CNS in rodents. Studies in humans, with proteins such as AVP, CCK analog, MSH/ACTH and insulin^{22,23} have revealed that they are delivered directly to the brain from the nasal cavity. Liu et al^{25,26} have demonstrated the therapeutic benefit of intranasal delivery of proteins in stroke studies. They have shown that intranasal IGF-I reduces infarct volume and improves neurologic function in rats with middle cerebral artery occlusion (MCAO)

Nasal absorption is affected by molecular weight, size, formulation pH, pKa of molecule, and delivery volume among other formulation characteristics. Molecular weight still presents the best correlation to absorption. The apparent cut-off point for molecular weight, is approximately 1,000 daltons, with molecules less than 1,000 having better absorption²⁴.

On this background the intranasal administration seems to be a promising option for pentinin delivery to the CNS.

Several patents or patent applications describe compositions administrated as nasal spray for the treatment of neurodegenerative diseases. However none of them describes pentinin or peptides similar to pentinin.

US20060039995 discloses methods and pharmaceutical compositions for preconditioning and/or providing neuroprotection to the animal central nervous system against the effects of ischemia, trauma, metal poisoning and neurodegeneration, including the associated cognitive, behavioral and physical impairments. Unlike the invention herein, the method is accomplished by stimulating and stabilizing hypoxia-inducible factor-1α (HIF-1α). HIF-1α is known to provide a neuroprotective benefit under ischemic conditions and has no connections to the effects of pentinin of the invention herein.

US20050019268 A1 reveals a spray containing ubiquinone for the treatment of neural disorders and neurodegenerative diseases. The ubiquinones are coenzymes and not like in the invention herein small peptides derived from insulin.

The possibility to deliver a number of protein therapeutic agents to the CNS using intranasal delivery in a variety of species is already known in the art. However nobody has described insulin derived peptides, so it was a surprise when we showed that conditioned medium from undifferentiated adult NSPCs protects hippocampal neurons from NMDA induced excitotoxicity. One component of that medium, a peptide which we termed pentinin, contained a high proportion of its neuroprotective activity. These data not only imply the presence of a new neuroprotective compound in the brain, but also suggest a new role for undifferentiated neural stem/progenitor cells as modulators of lesions in the brain.

### SUMMARY OF THE INVENTION

To explore the ability of endogenous undifferentiated stem cells to positively modulate damage, we investigated whether medium conditioned by adult hippocampal stem/progenitor cells affected excitotoxic cell death in organotypic hippocampal slice cultures. We found that conditioned medium significantly reduced cell death following 24 h exposure to 10 μM NMDA, and that the level of neuroprotection was greater in the dentate gyrus, compared to pyramidal cells of the comis amonis. Mass spectrometric analysis of the conditioned medium allowed for the identification of a pentameric peptide fragment, which corresponded to residues 26-30 (tyr, thr, pro, lys, thr) of the insulin B chain, which we termed pentinin. In the presence of 100 pM synthetic pentinin, the number of neurons killed by NMDA-induced toxicity was markedly reduced in the dentate gyrus. This invention discloses that progenitors in the subgranular zone may convert exogenous insulin into a pentinin capable of protecting neighbouring neurons from excitotoxic injury. An intranasal delivery system for administration of pentinin is also described. Other objects and features of the inventions will be more fully apparent from the following disclosure and appended claims.

### DETAILED DESCRIPTION OF THE INVENTION AND PREFERRED EMBODIMENTS THEREOF

NMDA-Induced Excitotoxicity in OHSCs

Since the experiments of the invention herein relies on the uptake of the nuclear dye propidium iodide (PI) as a marker of cell death, we performed immunofluorescent analysis to confirm identity of PI labelled cells. OHSCs were maintained in N2 medium, or exposed to 5 μM or 10 μM NMDA in N2 medium for 24 hours, in the presence of propidium iodide (PI). Slices were fixed and stained for NeuN, a marker of mature neurons. In addition, Caspase3A immunoreactivity was used to indicate caspase-dependent apoptosis.

There was a low level of PI staining in control cultures, which was most pronounced in the dentate gyrus. NMDA increased PI staining in a concentration dependent manner. The vast majority of PI labelled cells was co-labelled with NeuN, except in the dentate gyrus, where a small proportion of PI⁺ cells were NeuN⁻. On the basis of latter experiments (see below), these are likely to be DCX⁺ immature neuronal precursors. Hence, NMDA-induced cell death was primarily mediated by excitatory neurotoxicity. Although Caspase3A immunoreactivity was detected, this was not colocalised with NeuN, was not NMDA-dependent and was mostly found on the surface of the slice. It is likely that these cells are dying through another mechanism, which relates to the organotypic culture, such as tissue loss at the air interface. These cells were also not PI-labelled, and hence did not affect the level of PI staining.

NSPCs Secrete Neuroprotective Factors

To test the neuroprotective qualities of test media, OHSCs were first preincubated with PI for 24 hours. Fluorescent photomicrographs were taken, and used to determine the level of background staining. OHSCs were then transferred to test media one hour before 10 μM NMDA was added. Photomicrographs were taken again after 24 hours of NMDA exposure, and the change in PI staining intensity was quantified.

Test media were N2 medium and medium conditioned by NSPCs, with or without GDNF added. GDNF has previously been shown to be neuroprotective in OHSCs, when given at high doses (50-100 ng/ml¹⁰).

Exposure to 10 μM NMDA caused a greater than three-fold increase in PI staining. Incubation in CM led to a 33% reduction in NMDA-induced PI staining. Although a low dose of GDNF (1 ng/ml) added to N2 medium (control medium) did not reduce excitotoxicity. However, when this dose was added to CM, PI staining was significantly reduced to control levels.

Neuroprotection Mediated by NSPCs is Region Dependent

It was noted whilst observing the photomicrographs that the different hippocampal regions showed selective vulnerability for NMDA excitotoxicity, as well as preferential neuroprotection by test media. The dentate gyrus showed a low relative increase in PI staining after NMDA-exposure. This increase was abolished in the presence of CM, even without addition of GDNF. The CA1 and CA3 regions exhibited high vulnerability to NMDA, which was partly ameliorated in the presence of CM. However, addition of GDNF was required to restore control levels of PI staining in both regions.

NSPCs Produce a Peptide Derived from Insulin—Pentinin

Our laboratory had previously analysed NSPC-conditioned medium by mass spectrometry¹¹. This study identified a number of proteins, which could potentially mediate neuroprotection. The effects of adding some of these candidates to unconditioned medium have been tested, however, no evidence of neuroprotection was observed in this model (results not shown). To further investigate the secreted components of the CM, we applied a different mass spectrometric analysis that was optimised for the detection of peptides and smaller proteins (ranging from approximately 700-7000 Da).

This mass spectrometric analysis of medium conditioned by NSPCs revealed a peptide with a mass identical to a loss of residues B26-B30, tyr, thr, pro, lys, thr, (SEQ ID NO: 1) in the COOH-terminal of the bovine-insulin β-chain evolving during culturing of the cells. To confirm the identity of this peptide, cells were cultured in medium where the human insulin was replaced with bovine insulin. The mass of the peptide shifted and corresponded to loss of the same residues in the β-chain of human insulin. Thus, the mass shift between the intact protein and its cleavage product was contingent on the origin of the insulin, and the consequent differences in amino acid sequences. These cleavage products were not present in the control media.

A literature survey revealed that this truncation of insulin may be produced as a result of the action of insulin degrading enzyme (IDE). This enzyme cleaves insulin at various sites, including residue 26 of the B chain¹², and this produces a truncated B chain, as well as a pentameric fragment. A peptide (GPE) cleaved from insulin-like growth factor—1 has been shown to have neuroprotective properties (Refs 14-16). Hence, we were interested in testing the effects of this pentameric peptide, which we called pentinin.

We confirmed that NSPCs express IDE using immunofluorescent staining. Immunoreactivity was seen in all cells, and had a perinuclear localisation. This is consistent with reports of IDE being a cytosolic enzyme¹³. The properties of pentinin were tested using a stable, synthetic peptide, which was applied to the OHSC model.

Pentinin Reduces Excitotoxic Cell Death

We tested the neuroprotective properties of pentinin by adding the synthetic peptide to unconditioned medium in our NMDA-induced excitotoxicity model. A dose response assay showed that 100 μM provided an effective dose (results not shown). This was sufficient the reduce excitotoxicity induced by both 5 mM and 10 mM NMDA.

Pentinin Protects Both Immature and Mature Neuronal Cells

To determine the cell types which were protected by pentinin, we fixed OHSCs and performed immunofluorescence for markers of neuronally committed progenitors (DCX) and neurons (NeuN). Cells in the dentate gyrus were counted, and the percentage of cells double-labelled for PI and each marker was determined. The percentage of cells immunoreactive for immature and mature neuronal markers, co-labelled with PI, were markedly reduced (86% and 64%, respectively) in the presence of 100 pM pentinin.

The Nasal Spray

The composition according to the invention is preferably a nasal spray, so that the administration of pentinin can be effected on an intranasal route. The spray according to the invention is useful, in particular, for the treatment of conditions as encountered in stroke.

### EXAMPLE 1

### Preparation of NSPC Cultures

The NSPCs used in this study were adult rat hippocampal progenitor cells (AHPs), the isolation of which has been previously described^{6,7}. Clonally-derived cells were received at passage 4 as a gift from F. Gage (Laboratory of Genetics, The Salk Institute, La Jolla, Calif.). The cells were cultured in N2 medium (Dulbecco's modified Eagle's medium/Nut Mix F12 (1:1), 2 mM L-glutamine and 1% N2 supplement; Life Technologies, Taby, Sweden), supplemented with 20 ng/ml human recombinant bFGF (PeproTech, London, England). This medium was also used as unconditioned control medium.

AHPs retain the potential to differentiate into the three neural lineages (neuronal, astrocytic and oligodendrocytic⁸) and have a stable phenotype in long-term culture, retaining identical immunocytological characteristics for more than 30 passages⁶. In this study cells were used between passages 5 and 20 postcloning. AHP conditioned medium was produced by seeding AHPs (5×10⁴ cells/cm²) on to poly-ornithine/laminin coated 24-well plates. Cells were grown for two days before medium was collected and filtered (0.22 μm). Penicillin/streptomycin (PEST; 25 U/ml) and PI (2 μM) were added immediately before use. For studies involving GDNF, recombinant protein was added to control medium or CM, at a final concentration of 1 ng/ml.

### EXAMPLE 2

### Preparation of OHSC Cultures

Rat organotypic hippocampal slice cultures (400 μm thick) were prepared from P9 Sprague-Dawley rats, using the method of Stoppini and coworkers⁹. OHSCs were cultured in slice medium (50% BME, 25% EBSS, 23% horse serum, 7.5 mg/ml D-glucose, 1 mM L-glutamine and 25 U/ml PEST) for 12-14 days before experiments commenced.

### EXAMPLE 3

### Determination of NMDA-Induced Excitotoxicity and Neuroprotection

OHSCs were transferred to test media one hour before exposure to 10 μM NMDA for 24 h. The degree of NMDA-induced excitotoxicity was determined by comparing propidium iodide (PI) uptake prior to exposure with that following exposure. Pictures were captured using a digital camera (Olympus DP50) coupled to an inverted fluorescence microscope (Olympus IX70), equipped with a red long-pass WG fluorescence filter. Uptake of PI was quantified as the mean pixel intensity of epifluorescence, over the whole slice, or in defined sub-regions (ImageJ v1.29x).

### EXAMPLE 4

### Characterization of Cell Death by Immunohistochemistry

To characterise cell death, OHSCs were cultured in N2 medium with different concentrations of NMDA (in the presence of PI). After 24 h, OHSCs were washed in PBS and fixed in 4% paraformaldehyde (overnight, 4° C.). OHSCs were blocked and permeabilised by incubation for two hours in PTS (0.1M sodium phosphate buffer, 0.3% triton X-100 and 1% donkey serum (Jackson Immunoresearch Laboratories Inc., West Grove, Pa.)) at room temperature (RT), then incubated overnight (rocking, 4° C.) with mouse anti-NeuN antibody (1:500, Chemicon International Inc, Temecula, Calif.), rabbit anti-Caspase3A antibody (1:250, Cell Signalling Technology), and goat anti doublecortin antibody (Dcx, 1:400, Santa Cruz Biotechnology, Santa Cruz, USA). After thorough washing (3×30 mins in PTS, rocking), OHSCs were incubated overnight (rocking, 4° C.) with donkey anti-mouse Alexa 647-conjugated antibody (1:800, Molecular Probes, Leiden, Netherlands), donkey anti-rabbit Alexa 488-conjugated antibody (1:800, Molecular Probes) and donkey anti-goat Alexa 488-conjugated antibody (1:800, Molecular Probes). OHSCs were washed thoroughly and mounted in Prolong Gold mounting medium (Molecular Probes). Colocalisation of PI and/or Caspase3A staining with NeuN and Dcx immunofluorescence was determined by confocal microscopy (Leica TCS SP2, Leica Microsystems AG, Wetzlar, Germany).

### EXAMPLE 5

### Expression of Insulin-Degrading Enzyme (IDE), Determination by Immunocytochemistry

To determine whether IDE is expressed in AHPs, cells were seeded, in N2 medium, onto polyornithine/laminin coated glass coverslips, at a density of 5.0×10⁴ cells/cm². After fixation (4% paraformaldehyde in PBS, 4° C., 10 min), cells were pre-incubated for 30 min with PBS containing 3% bovine serum albumin (BSA) and 0.05% saponin (Sigma-Aldrich, Sweden AB) at RT. Subsequently, cells were incubated with mouse anti-IDE antibody (1:250, Covance Research Products, Berkeley, USA) and rabbit anti-musashi antibody (1:250, Chemicon) for 1 h at RT in PBS containing 1% BSA and 0.05% saponin. Following three washes in PBS, cells were incubated for 1 h at RT with secondary antibodies: Alexa Fluor 488-conjugated goat anti-mouse (1:2000, Molecular Probes) and Alexa Fluor 555-conjugated goat anti-rabbit (1:2000, Molecular Probes) and the nuclear dye TO-PRO-3 (1:1000, Molecular Probes).

### EXAMPLE 6

### Mass Spectrometric Analysis

Neural stem/progenitor cells were cultured in N2 medium supplemented with 20 ng/ml human recombinant bFGF for 48 h. Conditioned medium was collected, centrifuged to remove cellular material and stored at −20° C. until the analysis was performed. In this experiment, the N2 supplement contained either bovine or human insulin. Samples of CM (50 μl) were desalted and concentrated using ZipTip™ C18 (Millipore, Bedford, Mass., USA) according to the supplier's instructions. Subsequently, the samples were eluted with 3 μl of matrix solution (50 mg/ml 2,5-dihydroxybenzonic acid (DHB, Sigma St. Louise, Mo.) in acetone:0.1% trifluoric acid in water (4:1 v/v)) directly onto the highly polished, stainless steel, sample probe and left to dry at ambient conditions. The matrix-assisted laser desorption/ionization (MALDI) analyses were performed using an upgraded Bruker Reflex II instrument (Bruker-Franzen Analytik, Bremen, Germany) equipped with a two-stage electrostatic reflectron, a delayed extraction ion source, a high-resolution detector and a 2 GHz digitizer. The spectra were acquired in reflectron mode. Calibration was performed externally by using a mixture of peptides with known masses. Calibrant peptides were Met-enkephalin, angiotensin II, gamma-MSH, ACTH 18-39, mellitin and insulin (Sigma).

### EXAMPLE 7

### Nasal Preparation

A nasal preparation comprised of pentinin can also take a variety of forms for administration in nasal drops, gel, ointment, cream, powder or suspension, using a dispenser or other device as needed. A variety of dispensers and delivery vehicles are known in the art, including single-dose ampoules, atomizers, nebulizers, pumps, nasal pads, nasal sponges, nasal capsules, and the like.

More generally, the preparation can take a solid, semi-solid, or liquid form. In the case of a solid form, the components may be mixed together by blending, tumble mixing, freeze-drying, solvent evaporation, co-grinding, spray-drying, and other techniques known in the art.

A semi-solid preparation suitable for intranasal administration can take the form of an aqueous or oil-based gel or ointment. For example, pentinin can be mixed with microspheres of starch, gelatin, collagen, dextran, polylactide, polyglycolide, or other similar materials that are capable of forming hydrophilic gels. The microspheres can be loaded with drug, and upon administration form a gel that adheres to the nasal mucosa.

In a preferred embodiment, the nasal preparation is in liquid form, which can include an aqueous solution, an aqueous suspension, an oil solution, an oil suspension, or an emulsion, depending on the physicochemical properties of the composition components. The liquid preparation is administered as a nasal spray or as nasal drops, using devices known in the art, including nebulizers capable of delivering selected volumes of formulations as liquid-droplet aerosols. For example, a commercially available spray pump with a delivery volume of 50 μL or 100 μL is available from, for example, Valois (Congers, N.Y.) with spray tips in adult size and pediatric size.

The liquid preparation can be produced by known procedures. For example, an aqueous preparation for nasal administration can be produced by dissolving, suspending, or emulsifying the pentinin peptides in water, buffer, or other aqueous medium, or in a oleaginous base, such as a pharmaceutically-acceptable oil like olive oil, lanoline, silicone oil, glycerine fatty acids, and the like.

It will be appreciated that excipients necessary for formulation, stability, and/or bioavailability can be included in the preparation. Exemplary excipients include sugars (glucose, sorbitol, mannitol, sucrose), uptake enhancers (chitosan), thickening agents and stability enhancers (celluloses, polyvinyl pyrrolidone, starch, etc.), buffers, preservatives, and/or acids and bases to adjust the pH, and the like.

While the invention has been described with reference to specific embodiments, it will be appreciated that numerous variations, modifications, and embodiments are possible, and accordingly, all such variations, modifications, and embodiments are to be regarded as being within the spirit and scope of the invention.

### REFERENCES

1. Taupin, P. & Gage, F. H. Adult neurogenesis and neural stem cells of the central nervous system in mammals. J Neurosci Res 69, 745-9 (2002).
2. van Praag, H. et al. Functional neurogenesis in the adult hippocampus. Nature 415, 1030-4 (2002).
3. Ishibashi, S. et al. Human neural stem/progenitor cells, expanded in long-term neurosphere culture, promote functional recovery after focal ischemia in Mongolian gerbils. J Neurosci Res 78, 215-23 (2004).
4. Zhang, Z. G. et al. Magnetic resonance imaging and neurosphere therapy of stroke in rat. Ann Neurol 53, 259-63 (2003).
5. Llado, J., Haenggeli, C., Maragakis, N. J., Snyder, E. Y. & Rothstein, J. D. Neural stem cells protect against glutamate-induced excitotoxicity and promote survival of injured motor neurons through the secretion of neurotrophic factors. Mol Cell Neurosci 27, 322-31 (2004).
6. Gage, F. H. et al. Survival and differentiation of adult neuronal progenitor cells transplanted to the adult brain. Proc Natl Acad Sci USA 92, 11879-83 (1995).
7. Palmer, T. D., Takahashi, J. & Gage, F. H. The adult rat hippocampus contains primordial neural stem cells. Mol Cell Neurosci 8, 389-404 (1997).
8. Song, H., Stevens, C. F. & Gage, F. H. Astroglia induce neurogenesis from adult neural stem cells. Nature 417, 39-44 (2002).
9. Stoppini, L., Buchs, P. A. & Muller, D. A simple method for organotypic cultures of nervous tissue. J Neurosci Methods 37, 173-82 (1991).
10. Bonde, C. et al. GDNF and neublastin protect against NMDA-induced excitotoxicity in hippocampal slice cultures. Neuroreport 11, 4069-73 (2000).
11. Dahl, A. et al. Proteome analysis of conditioned medium from cultured adult hippocampal progenitors. Rapid Commun Mass Spectrom 17, 2195-202 (2003).
12. Stentz, F. B., Kitabchi, A. E., Schilling, J. W., Schronk, L. R. & Seyer, J. M. Identification of insulin intermediates and sites of cleavage of native insulin by insulin protease from human fibroblasts. J Biol Chem 264, 20275-82 (1989).
13. Akiyama, H. et al. Cellular localization of insulin-degrading enzyme in rat liver using monoclonal antibodies specific for this enzyme. Biochem Biophys Res Commun 155, 914-22 (1988).
14. Guan, J. et al. Neuroprotective effects of the N-terminal tripeptide of insulin-like growth factor-1, glycine-proline-glutamate (GPE) following intravenous infusion in hypoxic-ischemic adult rats. Neuropharmacology 47, 892-903 (2004).
15. Guan, J. et al. N-terminal tripeptide of IGF-1(GPE) prevents the loss of TH positive neurons after 6-OHDA induced nigral lesion in rats. Brain Res 859, 286-92 (2000).
16. Saura, J. et al. Neuroprotective effects of Gly-Pro-Glu, the N-terminal tripeptide of IGF-1, in the hippocampus in vitro. Neuroreport 10, 161-4 (1999).
17. Dluzniewska, J. et al. A strong neuroprotective effect of the autonomous C-terminal peptide of IGF-1 Ec (MGF) in brain ischemia. Faseb J 19, 1896-8 (2005).
18. Duckworth, W. C., Bennett, R. G. & Hamel, F. G. Insulin degradation: progress and potential. Endocr Rev 19, 608-24 (1998).
19. Fischer, W. H., Saunders, D., Brandenburg, D., Wollmer, A. & Zahn, H. A shortened insulin with full in vitro potency. Biol Chem Hoppe Seyler 366, 521-5 (1985).
20. Kuo, W. L., Montag, A. G. & Rosner, M. R. Insulin-degrading enzyme is differentially expressed and developmentally regulated in various rat tissues. Endocrinology 132, 604-11 (1993).
21. Talegaonkar S, Mishra P R. Intranasal delivery: An approach to bypass the blood brain barrier. Indian J Pharmacol 2004; 36:140-147).
22. Kern W, Born J, Schreiber H, Fehm H L. Central nervous system effects of intranasally administered insulin during euglycemia in men. Diabetes 1999; 48:557-63.
23. Hinchcliffe M, Illum L. Intranasal insulin delivery and therapy. Adv Drug Deliv Rev 1999; 35:199-234.
24. Wheatly M A, Dent J, Wheeldon E B, Smith P L. Nasal drug delivery: An in vitro characterization of transepithelial electrical properties and fluxes in the presence or absence of enhancer. J Control Release 1988; 8:167-77
25. Liu X F, Fawcett J R, Thorne R G, DeFor T A, Frey W H. Intranasal administration of insulin-like growth factor-1 bypass the blood brain barrier and protects against focal cerebral ischemic damage. J Neuro Sci 2001; 187:91-7.
26. Liu X F, Fawcett J R, Thorne R G, Frey W H 2nd. Non-invassive intranasal insulin-like growth factor-1 reduces infarct volume and improves neurologic function in rats following middle cerebral artery occlusion. Neurosci Lett 2001; 308: 914.

What is claimed is:

1. A peptide having neuroprotective properties comprising the pentameric peptide fragment tyr, thr, pro, lys, thr (SEQ ID NO: 1).

2. A method of affecting endogenous undifferentiated stem cells to positvely modulate neural damage comprising intranasally administering the peptide of claim 1 to a patient.

3. A method of treating disorders of the neural system comprising intranasally administering the peptide of claim 1 to a patient.

4. A method of formulating a medicine, comprising:
a) providing a peptide according to claim 1; and
b) formulating the peptide in a form selected from the group consisting of solid, semi-solid, and liquid.

5. The method of claim 4, further comprising including an excipient in the medicine.

6. The method of claim 4, wherein the medicine is in liquid form selected from the group consisting of an aqueous liquid and an oleaginous liquid.

7. The method of claim 4, wherein the formulation is an intranasal delivery system.

8. The method of claim 7, wherein the intranasal delivery system is a spray.

9. The method of claim 4, wherein the form is a semi-solid preparation for intranasal administration.

10. The method of claim 9, wherein the semi-solid preparation comprises microspheres of a material capable of forming a hydrophilic gel containing the peptide of claim 1.

11. A formulation for intranasal administration to a patient, comprising the peptide of claim 1.
